(19) United States
(12) Patent Application Publication
(10) Pub. No.: US 2026/0226414 A1
(43) Pub. Date: Aug. 6, 2026

(54) COMPOSITION FOR INDUCING DIFFERENTIATION OF MESENCHYMAL STEM CELLS INTO ADIPOCYTES

(71) Applicant: ENCELL CO., LTD, Seoul (KR)

(72) Inventors: Hong Bae JEON, Seoul (KR); Sun Jeong KIM, Seoul (KR); Soojin KWON, Seoul (KR); Jae Hoon SONG, Seoul (KR)

(21) Appl. No.: 19/153,325

(22) PCT Filed: Feb. 1, 2024
(86) PCT No.: PCT/KR2024/001564

§ 371 Date: Aug. 2, 2025

(30) Foreign Application Priority Data
Feb. 2, 2023 (KR) 10-2023-0014141

(51) Int. Cl. C12N 5/077

(52) U.S. Cl. CPC C12N 5/0653; C12N 2501/999; C12N 2506/1353; C12N 2506/1369; C12N 2506/1384

(57) ABSTRACT

The present invention relates to: a method for differentiating mesenchymal stem cells into adipocytes, wherein mesenchymal stem cells are treated with nervonic acid; and a composition for inducing the differentiation of mesenchymal stem cells into adipocytes, the composition containing nervonic acid as an active ingredient. According to the present invention, mesenchymal stem cells can be differentiated into adipocytes with high efficiency, and thus the present invention is useful for producing adipocytes used in procedures for ameliorating skin wrinkles.

### TECHNICAL FIELD

The present invention relates to a method of differentiating mesenchymal stem cells into adipocytes, and more particularly, to a method of differentiating mesenchymal stem cells into adipocytes including treating mesenchymal stem cells with nervonic acid and a composition for inducing adipogenesis of stem cells containing nervonic acid as an active ingredient.

### BACKGROUND ART

Fat grafting is a currently widely available method to improve skin elasticity. Fat grafting is a surgery to extract fat of a subject using a syringe and transplant the same to various areas of the skin that appear to have lost elasticity. This type of fat grafting has shown good results in reducing wrinkles in the skin due to aging, as well as in correcting lip lines, jaw lines, and forehead lines, rhinoplasty, and filling in areas where skin has sunken due to burns or injuries, or areas lost due to cancer surgery. However, it has been reported that 40-60% of the transplanted volume is reabsorbed into the human body after fat grafting (S Eremia et al., Dermatol. Sur. 2000, 26:1150-1158; Fulton J E et al., Dermatol. Clin. 2001, 19(3): 523-530), and fat grafting in areas with thin skin and low subcutaneous fat raises problems such as palpable cell clumps, and in fact, there are cases where the survival rate and survival period of the transplanted fat fall short of expectations, requiring re-grafting. Hence, there is an increasing demand for a composition capable of sufficiently increasing the subcutaneous adipose layer while replacing the fat grafting surgery that has these problems.

The basic structure of the skin is maintained by subcutaneous adipose tissue, and this subcutaneous adipose tissue plays a role in determining the volume and strength of the skin. Therefore, increasing the volume of adipose tissue may be a better solution for maintaining skin volume and reducing wrinkles than a conventional method of providing elasticity to the dermis or epidermis in the outer layer of the skin, as described above. Recently, studies related thereto have been published (Kim W S et al., J. Dermatol. Sci. 2009, 53(2): 96-102; Trottier V et al., Stem Cells. 2008 26(10):2713-23; Park B S et al., Dermatol. Surg. 2008, 34 (10):1323-1326).

Thorough research using adipocytes is ongoing in various fields. Preadipocytes, which are commonly used, are widely useful cells because they have the property of differentiating into adipocytes by differentiation-inducing material such as insulin. In research using adipocytes, mesenchymal stem cells and adipose-derived stem cells are recently receiving attention. These stem cells may be differentiated using methods similar to those commonly used to differentiate preadipocytes into adipocytes (Xu Y et al., Exp. Cell Res. 2006, 312, 1856-1864), and are thus widely employed in adipogenesis studies (Pittenger M F et al., Science 1999, 284, 143-146).

Accordingly, the present inventors have made great efforts to develop a method of differentiating mesenchymal stem cells into adipocytes with high efficiency, and ascertained that, when human-derived mesenchymal stem cells are treated with nervonic acid, differentiation into adipocytes may be promoted, thus culminating in the present invention.

### DISCLOSURE

An object of the present invention is to provide a method of differentiating mesenchymal stem cells into adipocytes with high efficiency.

Another object of the present invention is to provide a composition for inducing adipogenesis of mesenchymal stem cells.

In order to accomplish the above objects, the present invention provides a method of differentiating mesenchymal stem cells into adipocytes including treating mesenchymal stem cells with nervonic acid.

The present invention also provides a composition for inducing adipogenesis of mesenchymal stem cells containing nervonic acid as an active ingredient.

### BRIEF DESCRIPTION OF DRAWINGS

FIG. 1 shows results confirming cell viability by treating human adipose-derived mesenchymal stem cells with nervonic acid at different concentrations for 72 hours.

FIG. 2 shows results of selecting an appropriate concentration by observing formation of oil droplets by treating human adipose-derived mesenchymal stem cells with nervonic acid at different concentrations for 72 hours.

FIG. 3 shows results confirming cell viability by treating human adipose-derived mesenchymal stem cells with nervonic acid at different concentrations for 7 days.

FIG. 4a shows results of observation by Oil Red O staining to select the appropriate concentration for the best differentiation after inducing adipogenesis by treating human adipose-derived mesenchymal stem cells with nervonic acid at different concentrations.

FIG. 4b shows results of Oil Red O staining quantified to select the appropriate concentration for the best differentiation after inducing adipogenesis by treating human adipose-derived mesenchymal stem cells with nervonic acid at different concentrations.

FIG. 5a shows results of observation by Oil Red O staining after inducing adipogenesis for 7 days by treating human adipose-derived mesenchymal stem cells (AD-MSCs) with 160 μM nervonic acid.

FIG. 5b shows results of Oil Red O staining quantified after inducing adipogenesis for 7 days by treating human adipose-derived mesenchymal stem cells (AD-MSCs) with 160 μM nervonic acid.

FIG. 6a shows results of observation by Oil Red O staining after inducing adipogenesis for 14 days by treating human adipose-derived mesenchymal stem cells (AD-MSCs) with 160 μM nervonic acid.

FIG. 6b shows results of Oil Red O staining quantified after inducing adipogenesis for 14 days by treating human adipose-derived mesenchymal stem cells (AD-MSCs) with 160 μM nervonic acid.

FIG. 7a shows results of observation by Oil Red O staining after inducing adipogenesis for 21 days by treating human adipose-derived mesenchymal stem cells (AD-MSCs) with 160 μM nervonic acid.

FIG. 7b shows results of Oil Red O staining quantified after inducing adipogenesis for 21 days by treating human adipose-derived mesenchymal stem cells (AD-MSCs) with 160 μM nervonic acid.

FIG. 8a shows results of observation by Oil Red O staining after inducing adipogenesis by treating human umbilical cord blood-derived mesenchymal stem cells with 160 μM nervonic acid.

FIG. 8b shows results of Oil Red O staining quantified after inducing adipogenesis by treating human umbilical cord blood-derived mesenchymal stem cells with 160 μM nervonic acid.

FIG. 9a shows results of observation by Oil Red O staining after inducing adipogenesis by treating human placenta-derived mesenchymal stem cells with 160 μM nervonic acid.

FIG. 9b shows results of Oil Red O staining quantified after inducing adipogenesis by treating human placenta-derived mesenchymal stem cells with 160 μM nervonic acid.

FIG. 10a shows results of observation by Oil Red O staining after inducing adipogenesis by treating human umbilical cord-derived mesenchymal stem cells with 160 μM nervonic acid.

FIG. 10b shows results of Oil Red O staining quantified after inducing adipogenesis by treating human umbilical cord-derived mesenchymal stem cells with 160 μM nervonic acid.

FIG. 11a shows results of observation by Oil Red O staining after inducing adipogenesis by treating human bone marrow-derived mesenchymal stem cells with 160 μM nervonic acid.

FIG. 11b shows results of Oil Red O staining quantified after inducing adipogenesis by treating human bone marrow-derived mesenchymal stem cells with 160 μM nervonic acid.

FIG. 12 shows results confirming the expression of adipogenesis markers at the mRNA level through qRT-PCR after inducing adipogenesis for 14 days by treating human adipose-derived mesenchymal stem cells with 160 μM nervonic acid.

FIG. 13a shows results confirming the expression of adipogenesis markers at the protein level through Western blotting after inducing adipogenesis for 14 days by treating human adipose-derived mesenchymal stem cells with 160 μM nervonic acid.

FIG. 13b shows results of Western blotting quantified for the expression of adipogenesis markers at the protein level after inducing adipogenesis for 14 days by treating human adipose-derived mesenchymal stem cells with 160 μM nervonic acid.

FIG. 14a shows results confirming the expression of adipogenesis-associated signaling molecule markers at the protein level through Western blotting after inducing adipogenesis for 14 days by treating human adipose-derived mesenchymal stem cells with 160 μM nervonic acid.

FIG. 14b shows results of Western blotting quantified for the expression of adipogenesis-associated signaling molecule markers at the protein level after inducing adipogenesis for 14 days by treating human adipose-derived mesenchymal stem cells with 160 μM nervonic acid.

### MODE FOR INVENTION

Unless otherwise defined, all technical and scientific terms used herein have the same meanings as typically understood by those skilled in the art to which the present invention belongs. In general, the nomenclature used herein is well known in the art and is typical.

The present inventors sought to develop a method of effectively differentiating mesenchymal stem cells (MSCs) into adipocytes, confirming that, when human-derived mesenchymal stem cells are treated with nervonic acid, the mesenchymal stem cells are differentiated into adipocytes with high efficiency.

Accordingly, an aspect of the present invention relates to a method of differentiating mesenchymal stem cells into adipocytes, including treating mesenchymal stem cells with nervonic acid.

As used herein, the term “adipogenesis” means that preadipocytes or mesenchymal stem cells of an animal, preferably a mammal, differentiate into adipocytes, and more preferably human mesenchymal stem cells, most preferably mesenchymal stem cells derived from human adipose tissue, umbilical cord blood, placenta, umbilical cord, or bone marrow differentiate into adipocytes.

As used herein, the term “differentiation” refers to a phenomenon in which cells become specialized in structure or function while growing through division and proliferation, that is, cells, tissues, etc. of a living organism change in shape or function in order to perform respective assigned tasks thereof.

In the present invention, nervonic acid may be used at a concentration of 1 to 280 μM, preferably 40 to 280 μM, more preferably 80 to 240 μM, even more preferably 120 to 200 μM.

Another aspect of the present invention relates to a composition for inducing adipogenesis of mesenchymal stem cells containing nervonic acid as an active ingredient.

The composition of the present invention is capable of promoting differentiation of mesenchymal stem cells into adipocytes, thereby forming a subcutaneous adipose layer or providing adipocytes, ultimately exhibiting the effect of imparting volume and elasticity to the skin and reducing wrinkles, and may be provided as a skin external application composition or a pharmaceutical composition.

A better understanding of the present invention may be obtained through the following examples. These examples are merely set forth to illustrate the present invention and are not to be construed as limiting the scope of the present invention, as will be apparent to those skilled in the art.

### Example 1. Confirmation of Promotion of Differentiation of Human Adipose-Derived Mesenchymal Stem Cells into Adipocytes after Culture by Exposure to Nervonic Acid

The efficacy of promoting differentiation into adipocytes by treating human adipose-derived mesenchymal stem cells with nervonic acid was confirmed.

Adipose tissue-derived mesenchymal stem cells (AD-MSCs) were isolated from adipose tissue separated according to the previously reported method of Palumbo et al. (Int. J. Mol. Sci. (2018) 19: 1987).

The obtained adipose tissue-derived mesenchymal stem cells (AD-MSCs) were subcultured in aMEM (a-modified Minimum Essential Media) containing 10% fetal bovine serum (FBS) and 0.5% gentamicin (10 mg/ml).

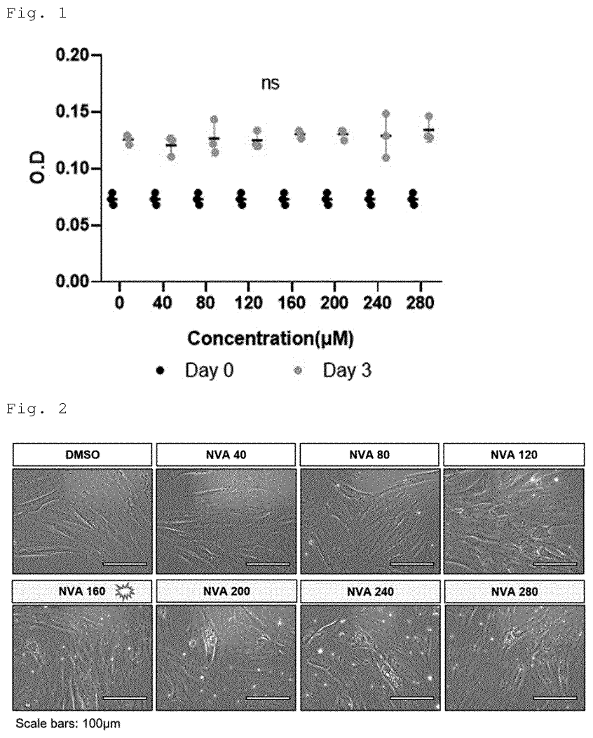

The isolated human adipose-derived mesenchymal stem cells were treated with nervonic acid at different concentrations and cultured for 72 hours, after which cytotoxicity was measured. Specifically, Cell Counting Kit-8 (CCK-8, Dojindo, Tokyo, Japan) was used, and 1×10³/well of cells were plated on a 96-well plate, stabilized for 24 hours, and then treated with nervonic acid at different concentrations (0, 40, 80, 120, 160, 200, 240, 280 μM), including the control group (DMSO), and cell viability was measured. Thereby, as shown in FIG. 1, there was no significant difference in cell viability in the nervonic acid treatment group after 3 days, confirming no cytotoxicity.

Human adipose-derived mesenchymal stem cells were treated with nervonic acid at different concentrations for 72 hours and cultured, after which the concentration at which oil droplets were generated was observed. Thereby, as shown in FIG. 2, oil droplets were observed at concentrations of 160 μM or higher, based on which adipogenesis was judged to be induced.

Before confirming the efficacy of nervonic acid to promote adipogenesis, cell viability was reconfirmed by treating cells with nervonic acid at various concentrations for 7 days. The specific method is the same as the method described above, and cell viability was measured by treatment with nervonic acid at different concentrations (0, 40, 80, 120, 160, 200, 240, 280 μM), including the control group (DMSO).

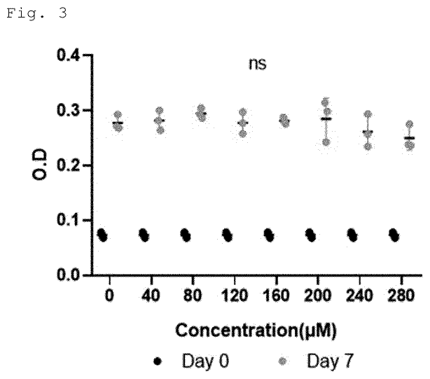

Thereby, as shown in FIG. 3, there was no significant difference in cell viability in the nervonic acid treatment group compared to the DMSO control group after 7 days, confirming no cytotoxicity.

### Example 2. Confirmation of Efficacy of Promoting Differentiation into Adipocytes after Inducing Adipogenesis by Treating Human Adipose-Derived Mesenchymal Stem Cells with Nervonic Acid

To determine the appropriate concentration of nervonic acid having excellent efficacy in promoting adipogenesis, adipogenesis was induced for 7 days by treating human adipose-derived mesenchymal stem cells with nervonic acid at different concentrations (0, 40, 80, 120, 160, 200, 240, 280 μM), followed by Oil Red O staining to confirm the stained oil droplets.

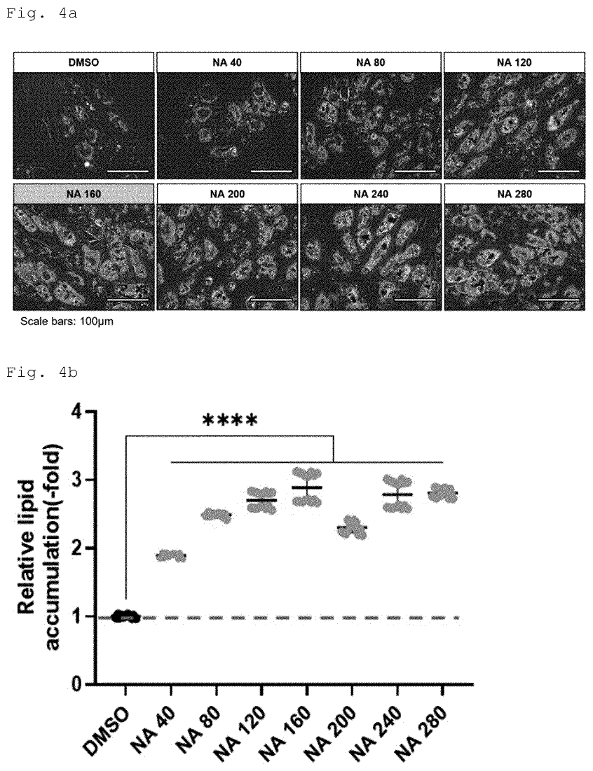

Specifically, for Oil Red O (Sigma-Aldrich, St. Louis, MO, USA) staining, after completion of differentiation, the culture medium was removed from each well of the plate, followed by washing twice with 1×PBS and then fixing with 4% paraformaldehyde (PFA) for 10 minutes. Washing three times with deionized water and then washing once with 60% isopropyl alcohol were performed. The Oil Red O stock solution was diluted with deionized water in a ratio of 3:2 to prepare an Oil Red O working solution, 1 ml of which was then added to each well of the plate followed by reaction at room temperature for 30 minutes. After washing once with 60% isopropyl alcohol and washing three times with deionized water, the red-stained oil droplets were observed and photographed under a microscope, and the results are shown in FIG. 4a.

After inducing adipogenesis for 7 days by treating human adipose-derived mesenchymal stem cells with nervonic acid at different concentrations (0, 40, 80, 120, 160, 200, 240, 280 μM), the results of quantification of the oil droplets stained by Oil Red O staining are shown in FIG. 4b.

Specifically, the stained oil droplets were dissolved in 100% isopropyl alcohol, and 100 μl of the result was transferred to each well of the 96-well plate, followed by measurement using a microplate reader (absorbance at 540 nm). Although adipogenesis was promoted from low to high concentrations, adipogenesis was most effectively induced at a concentration of 160 μM. Nervonic acid (Sigma-Aldrich, St. Louis, MO, USA) was dissolved in DMSO and then stored at −80° C. before use. In treatment with nervonic acid, the concentration of 160 μM, which does not affect cell viability and causes the best adipogenesis, was selected to confirm the efficacy of promoting adipogenesis.

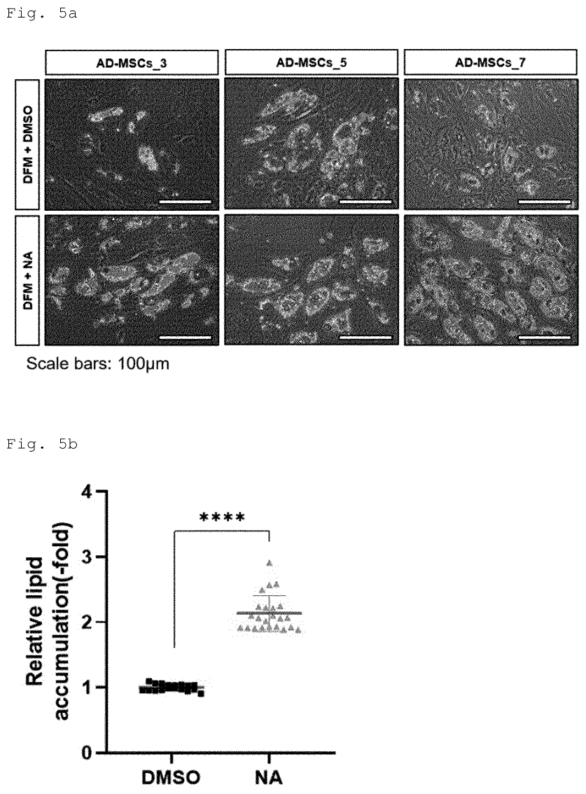

Adipogenesis was induced for 7 days by treating a total of three lots of human adipose-derived mesenchymal stem cells (AD-MSCs) with 160 μM nervonic acid, followed by observation of the oil droplets stained by Oil Red O staining, and the results are shown in FIG. 5a.

After inducing adipogenesis for 7 days by treating a total of three lots of human adipose-derived mesenchymal stem cells (AD-MSCs) with 160 μM nervonic acid, the results of quantification of the oil droplets stained by Oil Red O staining are shown in FIG. 5b, confirming that the nervonic acid-treated differentiation group showed a 2.13±0.27-fold increase compared to the DMSO-treated control group.

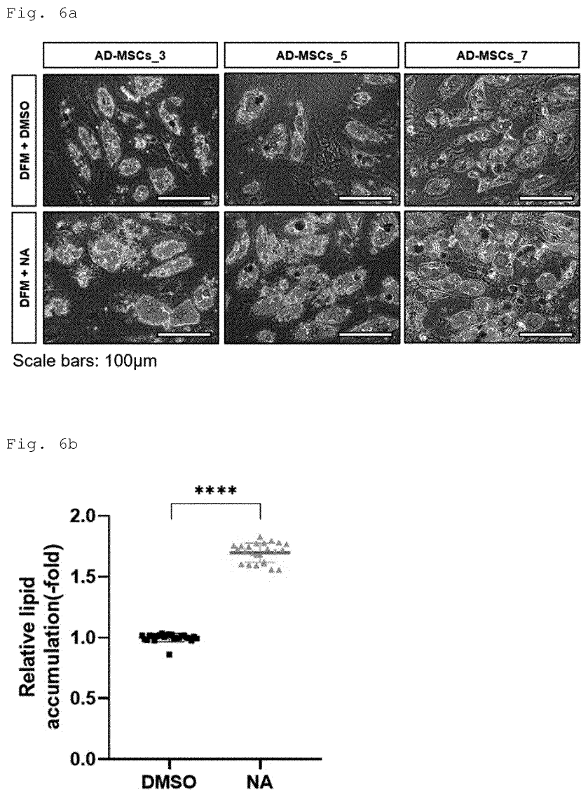

Adipogenesis was induced for 14 days by treating a total of three lots of human adipose-derived mesenchymal stem cells (AD-MSCs) with 160 μM nervonic acid, followed by observation of the oil droplets stained by Oil Red O staining, and the results are shown in FIG. 6a.

After inducing adipogenesis for 14 days by treating a total of three lots of human adipose-derived mesenchymal stem cells (AD-MSCs) with 160 μM nervonic acid, the results of quantification of the oil droplets stained by Oil Red O staining are shown in FIG. 6b, confirming that the nervonic acid-treated differentiation group showed a 1.70±0.08-fold increase compared to the DMSO-treated control group.

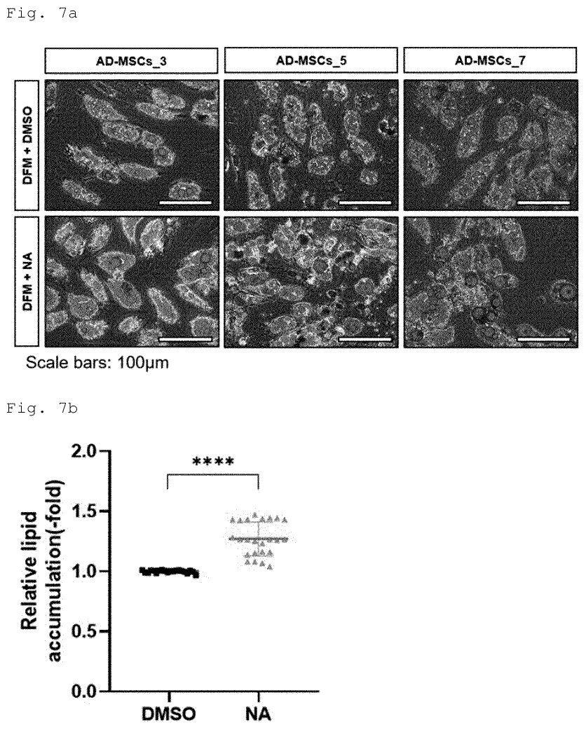

Adipogenesis was induced for 21 days by treating a total of three lots of human adipose-derived mesenchymal stem cells (AD-MSCs) with 160 μM nervonic acid, followed by observation of the oil droplets stained by Oil Red O staining, and the results are shown in FIG. 7a.

After inducing adipogenesis for 21 days by treating a total of three lots of human adipose-derived mesenchymal stem cells (AD-MSCs) with 160 μM nervonic acid, the results of quantification of the oil droplets stained by Oil Red O staining are shown in FIG. 7b, confirming that the nervonic acid-treated differentiation group showed a 1.27±0.14-fold increase compared to the DMSO-treated control group.

### Example 3. Confirmation of Efficacy of Promoting Differentiation into Adipocytes after Inducing Adipogenesis by Treating Human Other Tissue-Derived Mesenchymal Stem Cells with Nervonic Acid

Human umbilical cord, placenta, and umbilical cord blood were collected with maternal consent after approval from the Institutional Review Board (IRB) of Samsung Medical Center, and adipose tissue was also collected with patient consent after approval from the IRB of Samsung Medical Center.

Human placenta-derived mesenchymal stem cells (PL-MSCs) were isolated from placentas separated according to the previously reported method of Choi et al. (PLoS ONE, (2017) 12(2): e0172642), umbilical cord blood-derived mesenchymal stem cells (UCB-MSCs) were isolated from the umbilical cord blood separated according to the method of Kim et al. (FEBS Letters, (2010) 584: 3601-3608), and umbilical cord-derived mesenchymal stem cells (WJ-MSCs) were isolated from the umbilical cord separated according to the method of Park et al. (Arch. Pharm. Res. (2016) 39: 1171-1179).

Human bone marrow-derived mesenchymal stem cells (BM-MSCs) were purchased from Cambrex (Lonza, #PT-2501).

Each of the obtained mesenchymal stem cells was subcultured in aMEM (a-modified Minimum Essential Media) containing 10% fetal bovine serum (FBS) and 0.5% gentamicin (10 mg/ml).

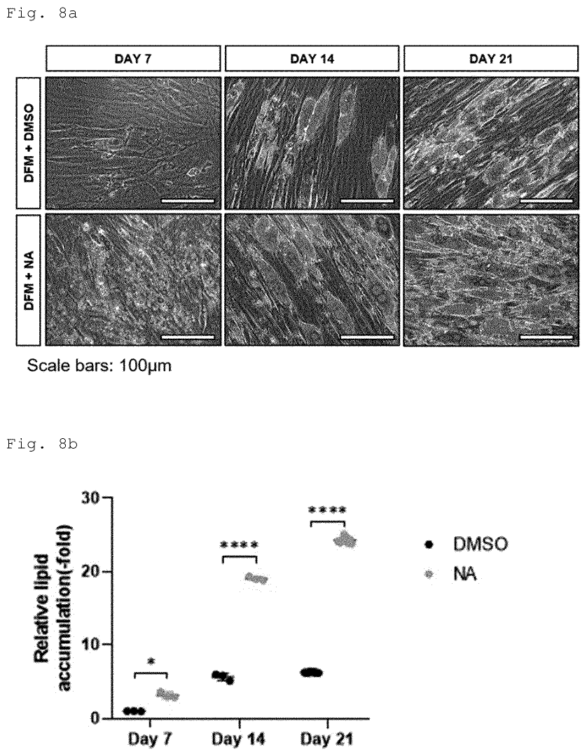

After inducing adipogenesis for 7, 14, and 21 days by treating human umbilical cord blood-derived mesenchymal stem cells (UCB-MSCs) with nervonic acid, the oil droplets stained by Oil Red O staining are shown in FIG. 8a, and the results of quantification of the stained oil droplets are shown in FIG. 8b. Thereby, compared to the DMSO control group, the nervonic acid-treated differentiation group showed a 2-fold or more increase in all cases, and the greatest 3.87±0.06-fold increase was confirmed on day 21.

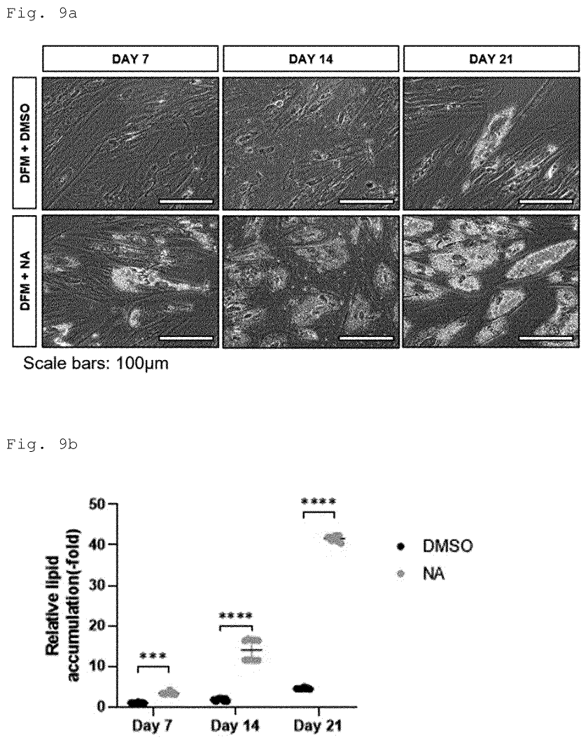

After inducing adipogenesis for 7, 14, and 21 days by treating human placenta-derived mesenchymal stem cells (PL-MSCs) with nervonic acid, the oil droplets stained by Oil Red O staining are shown in FIG. 9a, and the results of quantification of the stained oil droplets are shown in FIG. 9b. Thereby, compared to the DMSO control group, the nervonic acid-treated differentiation group showed a 2-fold or more increase in all cases, and the greatest 9.22±0.15-fold increase was confirmed on day 21.

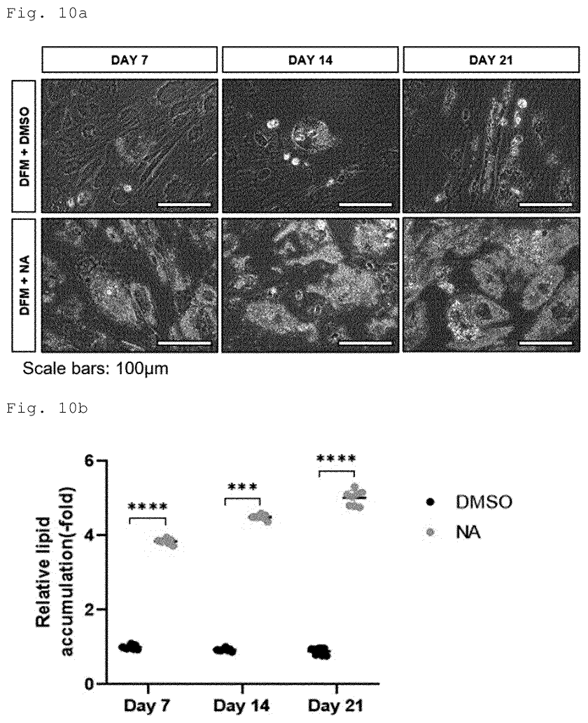

After inducing adipogenesis for 7, 14, and 21 days by treating human umbilical cord-derived mesenchymal stem cells (WJ-MSCs) with nervonic acid, the oil droplets stained by Oil Red O staining are shown in FIG. 10a, and the results of quantification of the stained oil droplets are shown in FIG. 10b. Thereby, compared to the DMSO control group, the nervonic acid-treated differentiation group showed a 2-fold or more increase in all cases, and the greatest 5.61±0.23-fold increase was confirmed on day 21.

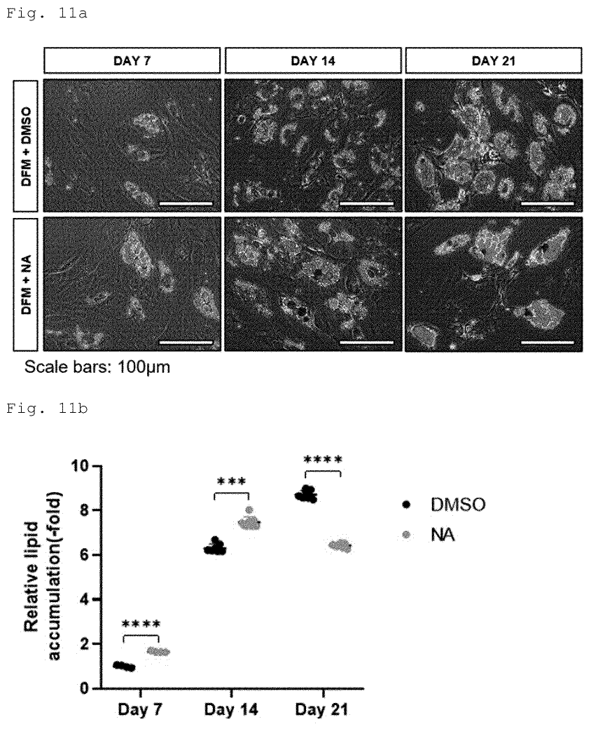

After inducing adipogenesis for 7, 14, and 21 days by treating human bone marrow-derived mesenchymal stem cells (BM-MSC) with nervonic acid, the oil droplets stained by Oil Red O staining are shown in FIG. 11a, and the results of quantification of the stained oil droplets are shown in FIG. 11b.

Thereby, adipogenesis slightly increased compared to the DMSO control group on days 7 and 14.

### Example 4. Comparison of Expression Levels of Adipogenesis-Associated Gene Markers after Inducing Adipogenesis by Treating Human Adipose-Derived Mesenchymal Stem Cells with Nervonic Acid

After inducing adipogenesis for 14 days by treating human adipose-derived mesenchymal stem cells (AD-MSCs) with 160 μM nervonic acid, the gene expression of adipogenesis markers at the mRNA level was compared using qRT-PCR.

Specifically, RNA for qRT-PCR was extracted from mesenchymal stem cells with adipogenesis completed using TRIZOL from Invitrogen according to the method provided by the vendor, and cDNA was synthesized using SuperScript™ IV Reverse Transcriptase from Invitrogen. qRT-PCR was performed using the primers listed in Table 1 below, and the quantified results through 2^{-ΔΔCt} analysis are shown in FIG. 12.

TABLE 1
Primer sequences used in qRT-PCR

| | Forward | Reverse |
|---|---|---|
| PPARγ | 5′-ATGGGTGAAAC | 5′-TGGAATGTCT |
| | TCTGGGAGA-3′ | TCGTAATGTGGA- |
| | (SEQ ID NO: 1) | 3′ |
| | | (SEQ ID NO: 2) |
| c/EBPa | 5′-ACTGGGACCCT | 5′-TGGACTGATC |
| | CAGCCTTG-3 | GTGCTTCGTG-3′ |
| | (SEQ ID NO: 3) | (SEQ ID NO: 4) |
| Adiponectin | 5′-AGATCCAGGTC | 5′-CTTTCCTGCC |
| | TTATTGGTCC-3′ | TTGGATTCC-3′ |
| | (SEQ ID NO: 5) | (SEQ ID NO: 6) |
| LPL | 5′-CATTCCCGGA | 5′-ATTCCTGTTA |
| | GTAGCAGAG-3′ | CCGTCCAGC-3′ |
| | (SEQ ID NO: 7) | (SEQ ID NO: 8) |
| Gapdh | 5′-GAAGGTGAAG | 5′-TGGCAACAATA |
| | GTCGGAGT-3′ | TCCACTTTACCA-3′ |
| | (SEQ ID NO: 9) | (SEQ ID NO: 10) |

Thereby, in the nervonic acid-treated differentiation group compared to the control group on day 14, the early transcription factors PPARγ and c/EBPa showed a 1.74±0.35-fold increase and a 1.66±0.22-fold increase, respectively, and the late mature adipocyte markers Adiponectin and LPL showed a 1.37±0.19-fold increase and a 1.44±0.12-fold increase, respectively.

### Example 5. Comparison of Protein Expression Levels of Adipogenesis Markers after Inducing Adipogenesis by Treating Human Adipose-Derived Mesenchymal Stem Cells with Nervonic Acid

After inducing adipogenesis for 14 days by treating human adipose-derived mesenchymal stem cells (AD-MSCs) with 160 μM nervonic acid, the expression of adipogenesis markers at the protein level was compared using Western blotting.

Specifically, protein for Western blotting was extracted from mesenchymal stem cells with adipogenesis completed using RIPA lysis buffer, and Western blotting was performed using the primary antibodies listed in Table 2 below.

TABLE 2
List of primary antibodies used in Western blotting

| | Manufacturer | Product number |
|---|---|---|
| PPARγ | Cell Signaling Technology | #2435 |
| C/EBPA | Cell Signaling Technology | #2295 |
| ADIPONECTIN | Cell Signaling Technology | #3288 |
| LPL | Santa Cruz Biotechnology | sc-373759 |
| β-actin | Santa Cruz Biotechnology | sc-47778 |

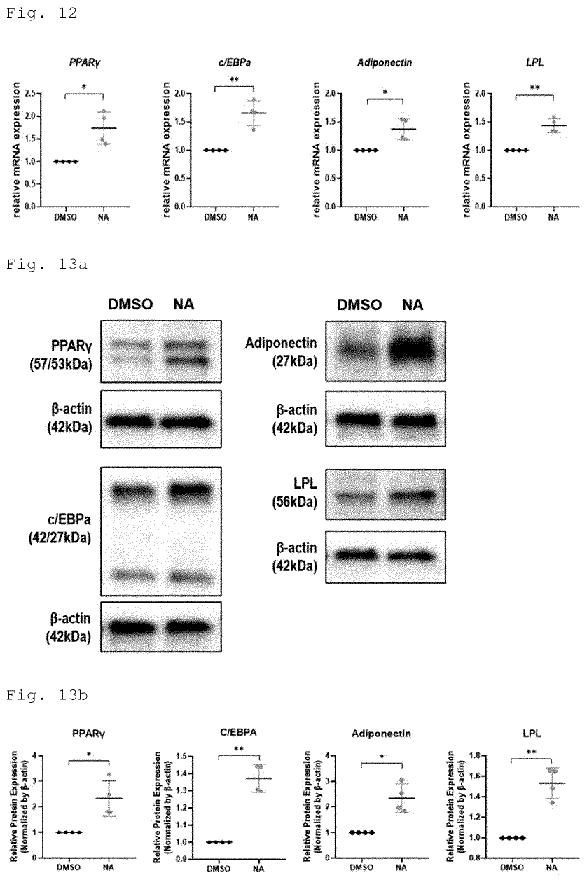

The results of protein expression confirmed through Western blotting are shown in FIG. 13a, and the results of quantification of the expression level of each marker are shown in FIG. 13b. Thereby, in the nervonic acid-treated differentiation group compared to the control group on day 14, the early transcription factors PPARγ and C/EBPA showed a 2.34±0.69-fold increase and a 1.37±0.08-fold increase, respectively, and the late mature adipocyte markers Adiponectin and LPL showed a 2.35±0.55-fold increase and a 1.53±0.15-fold increase, respectively.

### Example 6. Comparison of Protein Expression Levels of Adipogenesis-Associated Signaling Molecules after Inducing Adipogenesis by Treating Human Adipose-Derived Mesenchymal Stem Cells with Nervonic Acid

After inducing adipogenesis for 14 days by treating human adipose-derived mesenchymal stem cells (AD-MSCs) with 160 μM nervonic acid, the expression of adipogenesis-associated signaling molecules at the protein level was compared using Western blotting.

More specifically, protein for Western blotting was extracted using the same method as the extraction method described above, and Western blotting was performed using the primary antibodies listed in Table 3 below.

TABLE 3
List of primary antibodies used in Western blotting

| | Manufacturer | Product number |
|---|---|---|
| phospho-AKT | Cell Signaling Technology | #9271 |
| AKT | Cell Signaling Technology | #9272 |
| phospho-mTOR | Cell Signaling Technology | #5536 |
| mTOR | Cell Signaling Technology | #2983 |
| phospho-β- | Cell Signaling Technology | #9561 |
| catenin | | |
| β-catenin | Cell Signaling Technology | #9562 |
| β-actin | Santa Cruz Biotechnology | sc-47778 |

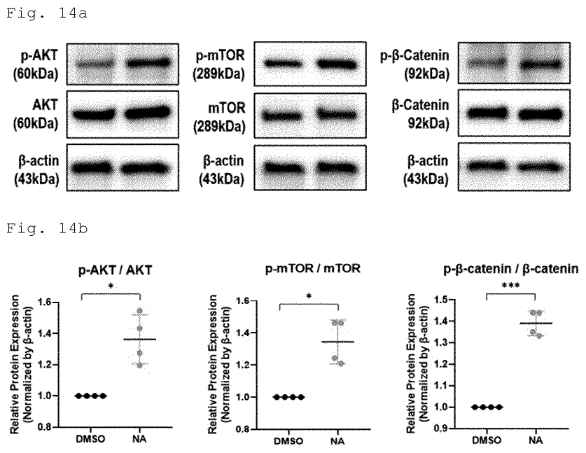

The results of protein expression confirmed through Western blotting are shown in FIG. 14a, and the results of quantification of the expression level of each signaling molecule are shown in FIG. 14b.

Thereby, in the nervonic acid-treated differentiation group compared to the control group on day 14, respective phosphorylation ratios, which are activation indicators of AKT and mTOR as signaling molecules that promote adipogenesis, showed a 1.36±0.16-fold increase and a 1.34±0.14-fold increase, and the phosphorylation ratio, which is an inactivation indicator of B-catenin as a signaling molecule that inhibits adipogenesis, showed a 1.39±0.06-fold increase.

Therefore, it is deemed that nervonic acid is usable in a composition for inducing differentiation by promoting adipogenesis of a variety of mesenchymal stem cells.

### INDUSTRIAL APPLICABILITY

The present invention is capable of differentiating mesenchymal stem cells into adipocytes with high efficiency and is therefore useful for producing adipocytes used in operations for reducing skin wrinkles.

Having described certain parts of the present invention in detail above, it will be obvious to those skilled in the art that these specific descriptions are only preferred embodiments, and the scope of the present invention is not limited thereby. Accordingly, the substantial scope of the present invention will be defined by the appended claims and equivalents thereto.

1. A method of differentiating mesenchymal stem cells into adipocytes, comprising treating mesenchymal stem cells with nervonic acid.

2. The method according to claim 1, wherein the nervonic acid is used at a concentration of 1 to 280 μM.

3. The method according to claim 1, wherein the mesenchymal stem cells are derived from a tissue selected from the group consisting of adipose tissue, umbilical cord blood, placenta, umbilical cord, and bone marrow.

4. A composition for inducing adipogenesis of mesenchymal stem cells, containing nervonic acid as an active ingredient.


SEQUENCE LISTING (10 sequences)

SEQ ID NO: 1 - DNA, 20 bp, synthetic construct
  source 1..20: mol_type = other DNA
atgggtgaaactctgggaga

SEQ ID NO: 2 - DNA, 22 bp, synthetic construct
  source 1..22: mol_type = other DNA
tggaatgtcttcgtaatgtgga

SEQ ID NO: 3 - DNA, 19 bp, synthetic construct
  source 1..19: mol_type = other DNA
actgggaccctcagccttg

SEQ ID NO: 4 - DNA, 20 bp, synthetic construct
  source 1..20: mol_type = other DNA
tggactgatcgtgcttcgtg

SEQ ID NO: 5 - DNA, 21 bp, synthetic construct
  source 1..21: mol_type = other DNA
agatccaggtcttattggtcc

SEQ ID NO: 6 - DNA, 19 bp, synthetic construct
  source 1..19: mol_type = other DNA
ctttcctgccttggattcc

SEQ ID NO: 7 - DNA, 19 bp, synthetic construct
  source 1..19: mol_type = other DNA
cattcccggagtagcagag

SEQ ID NO: 8 - DNA, 19 bp, synthetic construct
  source 1..19: mol_type = other DNA
attcctgttaccgtccagc

SEQ ID NO: 9 - DNA, 18 bp, synthetic construct
  source 1..18: mol_type = other DNA
gaaggtgaaggtcggagt

SEQ ID NO: 10 - DNA, 23 bp, synthetic construct
  source 1..23: mol_type = other DNA
tggcaacaatatccactttacca